(19) United States
(12) Patent Application Publication
(10) Pub. No.: US 2011/0015643 A1
(43) Pub. Date: Jan. 20, 2011

(54) LIGAMENT RECONSTRUCTION GUIDE ASSEMBLY AND METHODS OF USE

(76) Inventor: Lonnie E. Paulos, Pensacola, FL (US)

Correspondence Address: JOHN BROOKS LAW LLC, P.O. Box 156, WRENTHAM, MA, 02093, US

(21) Appl. No.: 12/934,280

(22) PCT Filed: Mar. 31, 2009
(86) PCT No.: PCT/US09/38885

§ 371 Date: Sep. 24, 2010

Related U.S. Application Data
(60) Provisional application No. 61/041,228, filed on Mar. 31, 2008.

(51) Int. Cl. A61B 17/56

(52) U.S. Cl. 606/96; 606/86 R

(57) ABSTRACT

The surgical guide assembly and methods of use utilize the geometric and trigonometric relationships of triangles to position bone tunnel positions for ligament reconstruction surgery. In one embodiment, the surgical guide assembly comprises a first guide pin and a guide frame having a first pin channel and a second pin channel, each channel having a channel longitudinal axis that is positioned at an angle to the other about a central convergence point. The assembly further includes a reference facility whereby placement of the first pin in the first pin channel predetermines the position of the longitudinal axis of the second pin channel relative to the first pin. In one embodiment, the position of the channels corresponds to the position of bone tunnels in a double-bundle ligament reconstruction procedure. Methods of using the guide assembly are also disclosed.

### CROSS-REFERENCE TO RELATED APPLICATIONS

This application claims the benefit of U.S. App. No. 61/041,228, filed on Mar. 31, 2008, entitled “ANTERIOR CRUCIATE LIGAMENT RECONSTRUCTION GUIDE ASSEMBLY AND METHOD OF USE,” the entire contents of which is incorporated herein by reference.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Field of the Invention

Embodiments of the present invention relate to assemblies and methods for use in reconstruction of ligaments and, more specifically, to assemblies and methods of locating bone tunnels in double bundle ligament reconstruction procedures.

2. Description of the Related Art

A common ligament reconstruction procedure is to replace a damaged ligament with an implant. Commonly, these implants are secured in the patient by creating a tunnel in the bone and that tunnel opens at the attachment site of the replacement implant. Common ligament reconstruction surgeries include reconstruction of the anterior cruciate ligament (ACL) and the posterior cruciate ligament (PCL) of the human knee.

Many ACL reconstructions used only one bundle of tissue as the ligament implant. However, the ACL is composed of two functional bundles, the anteromedial (AM) bundle and the posterolateral (PL) bundle. Therefore, in performing a single bundle procedure, the surgeon is forced to choose which anatomic bundle, and thus which function, he or she wishes to restore. As a result, some of the function of the ACL to stabilize the knee during different ranges of extension, flexion and rotation would be compromised in the post-op patient compared to the normal ACL. In most cases using other procedures, the bundle that is restored is the anteromedial bundle, and thus rotational stability and other load stability related to the posterolateral bundle would not be regained. The tendency for a single bundle reconstruction to be placed non-anatomic or to ignore the posterior lateral bundle of the ACL has also been noted in the art.

To support the single bundle procedure, single bundle ACL reconstruction guide devices exist in the art. These guides are used to locate and guide the creation of bone tunnels to hold and fix the substitute ligament. Typical single-bundle reconstruction assemblies and procedures are disclosed in U.S. Patent Pub. No. 2004/0193167 filed on Apr. 8, 2004 and entitled “TRANSVERSE SUSPENSION DEVICE” to Tucciarone et. al. and U.S. Patent Publication and U.S. Pat. No. 4,781,182 filed on Oct. 3, 1986 and entitled “APPARATUS AND METHOD FOR USE IN PERFORMING A SURGICAL OPERATION” to Purnell et. al. both of which are incorporated herein in their entirety.

Recent studies have been conducted which demonstrate better function of a double bundle ligament. The double bundle double tunnel procedure uses tendon autografts from local sources, or other suitable implants such as cadavers, to create two bundles that represent the two bands of the natural ACL. Two tunnels each are then drilled through both the femur and tibia for the attachment of each of the grafts using what is commonly called the transtibial technique. The ultimate goal of the double bundle double tunnel procedure, which is to restore both bundles of the ACL, both the anteromedial and the posterolateral.

For many double bundle procedures, the present ligament guide systems for a single bundle ACL surgery are used to create a tunnel for one bundle and the second bundle is placed using guide wires that are placed free hand. To do this it is necessary to remove the knee from its arthroscopic holding device and hyper-flex the knee and then pass a guide pin through a portal into the knee.

One assembly for positioning double bundle bone tunnels for ACL reconstruction, from an outside-in position, is disclosed in U.S. Pat. No. 5,324,295 to Michael Shaprio entitled “DRILL GUIDE FOR SURGICAL PINS” which is herein incorporated by reference in its entirety. This guide uses multiple holes in the guide to locate different convergence angles and therefore different tunnel locations in the patient's knee.

Reconstruction procedures for posterial cruciate ligament (PCL) surgeries have also started incorporating the double bundle procedure.

### BRIEF SUMMARY OF THE INVENTION

It is an object of one embodiment of the invention to provide a surgical guide assembly comprising a guide frame having a first pin channel and a second pin channel, each channel having a channel longitudinal axis, the longitudinal axis of each channel is positioned at an angle to the other about a central convergence point, a first pin capable of being received in the first pin channel and the assembly having a reference facility whereby placement of the first pin in the first pin channel predetermines the position of the longitudinal axis of the second pin channel.

It is a further object of one embodiment of the invention to provide a surgical guide assembly of claim 1 wherein the angle is between a range of about 10 to 55 degrees and the guide is used in positioning a bone tunnel in a double bundle ACL or double bundle PCL reconstruction.

It is another object of one embodiment of the invention to provide a surgical guide assembly wherein the second pin channel is capable of receiving a second pin and the assembly is used in a double bundle cruciate ligament reconstruction whereby the second pin locates a bone tunnel from an outside-in position.

It is an object of one embodiment of the invention to provide a reconstruction guide assembly wherein the reference facility comprises a frame reference point, a first pin reference point, a first pin end, and a predetermined reference length between the first pin reference point and the first pin end whereby the cooperation of the frame reference point with the pin reference point and the predetermined reference length of the first pin predetermines the position of the longitudinal axis of the second pin channel relative to the first pin end. In one embodiment, the reference facility further comprises a securing element wherein the first pin can be secured within the first pin channel of the guide frame.

It is another object of one embodiment of the invention to provide a reconstruction guide assembly comprising a guide frame having a first pin channel and a second pin channel, each channel having a channel longitudinal axis, the longitudinal axis of each channel being positioned at an angle to the other, a first rigid pin capable of being received in the first pin channel and the assembly having a reference facility whereby placement of the first rigid pin in the first pin channel predetermines the position of the longitudinal axis of the second pin channel.

It is a further object of one embodiment of the invention to provide the drill guide assembly where the angle is between a range of about 10 to 55 degrees and the guide is used in positioning a bone tunnel in an arthroscopic double bundle ACL reconstruction or arthroscopic double bundle PCL reconstruction.

It is still another object of one embodiment of the invention to provide the drill guide assembly where the reference facility comprises a frame reference point, a first pin reference point, a first pin end and a predetermined reference length between the first pin reference point and the first pin end whereby the cooperation of the frame reference point with the pin reference point and the predetermined reference length of the first pin predetermines the position of the longitudinal axis of the second pin channel relative to the first pin tip.

It is another object of one embodiment of the invention to provide the drill guide assembly where each channel longitudinal axis is defined by the radial center of the respective channel, the second pin channel is capable of receiving a second pin and the assembly is used in a double bundle cruciate ligament reconstruction whereby the second pin locates a bone tunnel created from an outside-in position.

It is an object of one embodiment of the invention to provide a method of locating a bone tunnel comprising: deploying a first pin with a longitudinal axis through a bone, receiving the first pin in a first pin channel of a guide frame, positioning a second pin with a longitudinal axis through a second pin channel of the guide frame, deploying the second pin through a bone and locating a first and second bone tunnel by the longitudinal axes of the first and second pin.

It is another object of one embodiment of the invention to provide a method of locating a bone tunnel where the method further comprises the first pin having a first pin reference point, the guide frame having a frame reference point and the step of receiving the first pin in a first pin channel of a guide frame further comprises engaging the first pin reference point with the frame reference point.

It is still another object of one embodiment of the invention to provide a method of locating a bone tunnel where the method further comprises the first pin channel having a first longitudinal axis, the second pin channel having a second longitudinal axis, the longitudinal axis of each channel is positioned at an angle to the other about a central convergence point and the step of locating the first and second bone tunnel further comprises positioning each tunnel at the angle relative to the central convergence point.

It is yet another object of one embodiment of the invention to provide a method of locating a bone tunnel where the method is used in a double-bundle ACL reconstruction procedure or a double-bundle PCL reconstruction procedure from an outside in position.

It is another object of one embodiment of the invention to provide the method of locating a bone tunnel further comprising the first pin having a first pin reference point, a first pin tip and a reference length between the first pin reference point and the first pin tip, the guide frame having a frame reference point and the step of receiving the first pin in a first pin channel of a guide frame further comprises engaging the first pin reference point with a frame reference point.

### BRIEF DESCRIPTION OF THE SEVERAL VIEWS OF THE DRAWINGS

In order that the manner in which the advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates a partial exploded front view of one embodiment of a guide frame of the reconstruction guide assembly.

FIG. 2 illustrates a front view of one embodiment of a guide frame showing channel placement.

FIG. 3 illustrates a side view of one embodiment of a guide pin.

FIG. 4 illustrates a side view of one embodiment the reconstruction guide assembly having a first and second pin installed.

FIG. 5 illustrates the dimensional relationships of one embodiment of the reconstruction guide assembly.

FIG. 6 illustrates a cut-away side view of the a femur and the resulting tunnels created using one embodiment of the guide assembly for creating femoral bone tunnels for a PCL reconstruction.

FIG. 7 illustrates a process flow diagram of one embodiment of the methods of using the reconstruction guide assembly.

FIG. 8 illustrates a front view of one embodiment of the invention used to place guide pins for a double-bundle ACL reconstruction.

### DETAILED DESCRIPTION OF THE INVENTION

Embodiments of a guide assembly and methods for their use will now be described in detail with reference to the accompanying drawings. It will be appreciated that, while the following description focuses on a guide assembly and methods that can be used in ACL reconstruction procedures, the systems and methods disclosed herein have wide applicability. For example, the guide assembly and methods described herein may be readily employed with PCL and other similar orthopedic procedures. The guide assembly can also be used in helping create bone tunnels in other reconstruction procedures such as in the rotator cuff. Notwithstanding the specific example embodiments set forth below, all such variations and modifications that would be envisioned by one of ordinary skill in the art are intended to fall within the scope of this disclosure.

### Ligament Reconstruction:

Single bundle ligament reconstruction procedures use a single graft to replace a damaged ligament. For example, when the ACL is reconstructed with this procedure, the graft is placed in the position of one ACL bundle, usually the anteromedial bundle. These grafts are held in place by initially making a bone tunnel through the bone. One tunnel is made in the femur and one in the tibia. With these procedures, the graft is secured in each bone tunnel with a fixation device such as a screw.

For a double bundle ligament reconstruction procedure, instead of placing just one larger graft, the double bundle ligament reconstruction procedure uses two smaller grafts that represent the anteromedial and the posterolateral ligament bundles of a normal ACL. Compared to the single bundle procedure, there are essentially two ligament reconstructions, one for each bundle. The double-bundle procedure requires two additional bone tunnels to accommodate the second bundle. The goal of this double bundle procedure is to more closely mimic the natural ligament construction and better restore the patient's range of motion within the knee and improved restoration of knee stability. However, the location of the tunnels can make them difficult to place. Manual positioning of the second tunnel typically lengthens the time for the operative procedure as well as increases the potential for contamination during surgery. Being able to place both bundles of the femoral ACL attachment without repositioning the knee is a great advantage.

The position of the anteromedial and the posterolateral ligament bundles of the ACL have a consistent geometric relationship relative to each other. By recognizing this geometric relationship, and configuring a complementary guide, the proper placement of the tunnels for these procedures can be made. By recognizing this relationship, it is also possible to create tunnels from a position outside of the knee joint and into the area of ligament connection to both the femur and the tibia that still maintains the positional relationship needed for proper ligament positioning. This ability to position tunnels from outside of the knee joint to the inside of the joint is referred to as an “outside-in” technique and it is performed from an “outside-in” position. One benefit of performing tasks from an outside-in position is that the knee does not always need to be repositioned as may be required to gain access to joint surfaces in an “inside-out” procedure.

Similar geometric relationships can be defined for a double bundle PCL femur connection.

### One Embodiment of the Reconstruction Guide Assembly

The ligament reconstruction guide assembly utilizes the geometric relationship of sides and angles of a triangle to position bone tunnels for surgical procedures. For example, when two sides of a triangle diverge from a central convergence point at a known angle, the relationship of the two sides will always be defined by that angle. These geometric rules parallel similar rules that can be applied to the positioning of bone tunnels to support double bundle ACL and PCL reconstructions. These relative attachment points of ligaments can be defined by each other because the femoral attachment sites are specific for both bundles of the ACL and PCL and are generally in the same relationship to each other regardless of gender, limb length or knee dimensions. Furthermore, using geometric and trigonometry relationships such as the Pythagorean Theorem, by defining a right triangle with the two sides of the triangle diverging from the central convergence point, and knowing the lengths of two sides of the triangle, you can determine the length of the third side. And if the triangle is not a right triangle, knowing dimensions such as angles and side lengths, the other elements of the triangle can be determined by common trigonometry relationships such as but not limited to the law of sines and/or cosines.

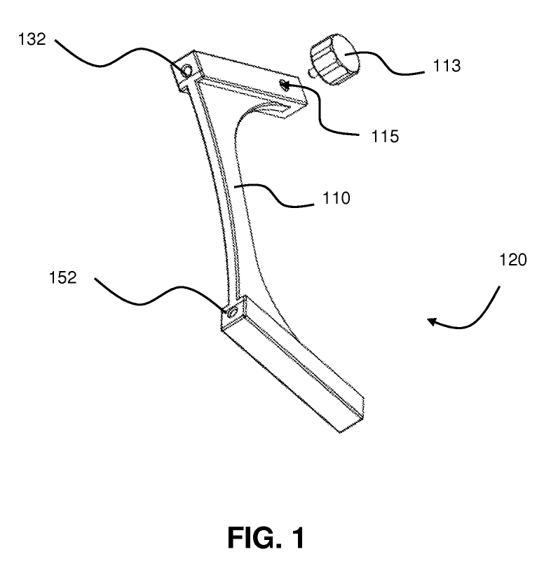

The reconstruction guide assembly comprises a guide frame and at least one pin. The guide frame comprises at least two fixed channels to receive the pins. In one embodiment, shown in FIG. 1, this guide frame 120 comprises a body 110 having a first and a second pin channel. The channels function as a means to guide elements such as guide pins and can comprise tunnels, slots, cooperating rings or other methods of guiding a reference elements such as an elongated rod. For illustration and not for limitation, the embodiment described below and in FIG. 1 the guide frame 120 comprises a body 110 having multiple tunnels to function as the channels. The distal end openings of the first and second tunnel are shown as 132 and 152 respectively. The dimensions of the body 110 may be any dimension suitable for the particular purpose for which they are used and is shaped to provide the proper relationship of the tunnels without being too large to use during the procedures. In the embodiment of FIG. 1, the body 110 has a length that provides for a tunnel length in the body of about 5 centimeters (cm), a height of about 5 cm and a thickness of about 2.5 cm. The body may be made of any rigid material that will maintain the relationship of the channels. These materials include but are not limited to steel, stainless steel, epoxy, composites, plastic or a combination of suitable surgical materials. Although embodiments are illustrated with the body being generally solid, it is understood that other structures such as wire frames or other skeletal structures can be suitable to provide the functionality and serve the purpose of the guide frame.

Although not required, the guide frame 120 in FIG. 1 further comprises a securing element 113 to secure the first guide pin in the first pin channel during use. As shown in FIG. 1, the securing element 113 is a threaded set knob that is received in a threaded channel 115. The securing element 113 is urged through the channel 115 and towards the center of the first pin channel through the use of the knob that can be frictionally turned by the hand of a user. When a guide pin is in the channel, the screw frictionally retains the pin in position in the channel. Although a set knob is used in this embodiment, any method of removably securing the first guide pin relative to the first channel is sufficient. Such methods include ball detents with a corresponding recess in the guide pin, restrictions in pin channel diameter that engage pin elements to restrict the movement of the pin relative to the pin channels, or other similar mating methods. Other methods of engaging the set screw are also contemplated such as a screw driver engaging a mating head of the set screw.

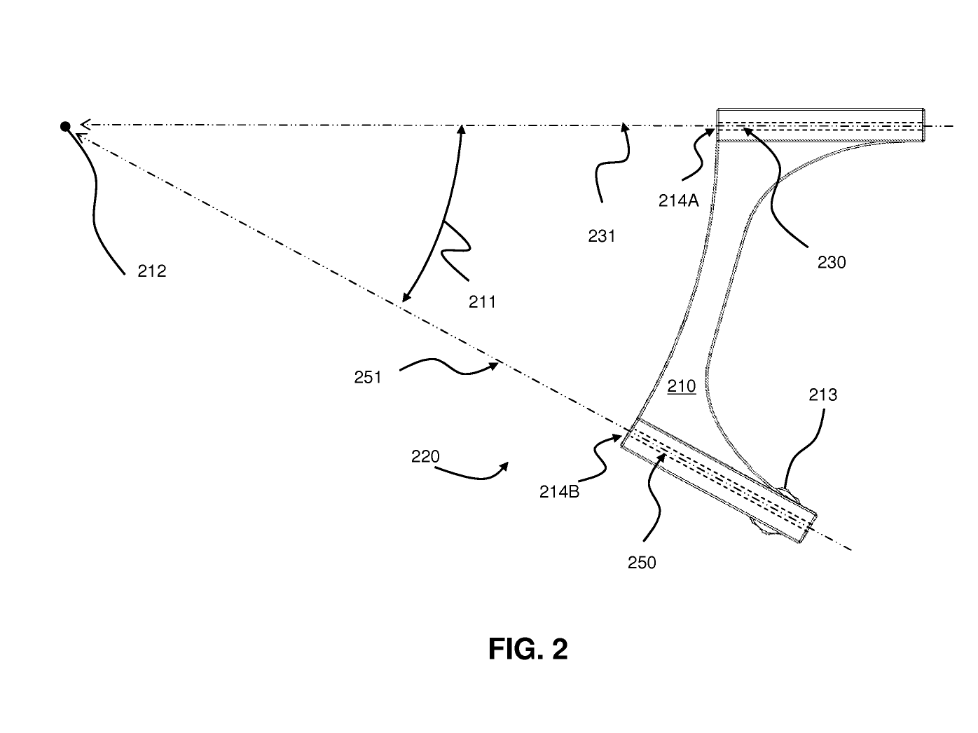

As shown in FIG. 2, the first pin channel 230 and the second pin channel 250 each have a central longitudinal axis 231 and 251 respectively having a radius about that longitudinal axis sized to receive the first pin and a second pin. The angle between the central longitudinal axis of the first pin channel 230 and second pin channel 250 from a central point 212 where these extended axes converge is defined by an angle represented in FIG. 2 as 211. The channels should be sized to allow pins to be received within the channel. The channels should be sized large enough to allow the pins to move in and out of the channel freely in a longitudinal direction, but they should be only slightly larger than the outer dimension of the pin so that the channel prevents the pins from having significant lateral movement within the channel.

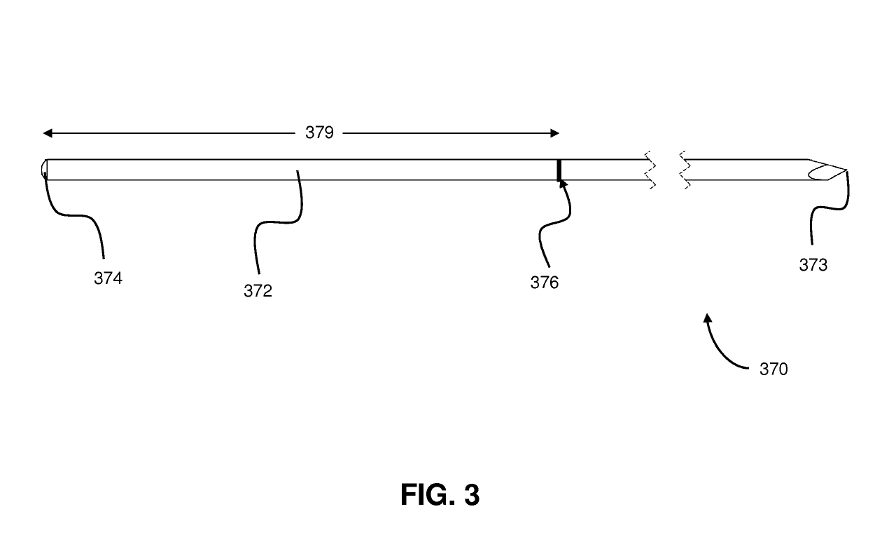

The first pin is an elongated rod and generally functions as a guide pin in common ACL or PCL reconstruction procedures. In one embodiment of the first pin shown in FIG. 3, the first pin 370 is an elongated rod 372 having a sharpened end 373 and a blunt end 374. The rod 372 may be any length or thickness suitable for the particular purpose they are used. In the embodiment illustrated in FIG. 3, the rod 372 length is about 30.5 cm in length and having a diameter of about 0.25 cm. The first pin 370 is generally rigid and made from surgical materials such as but not limited to titanium, stainless steel, nitinol, metal alloys, plastics or other suitable synthetic materials. A Kirschner wire (also called a K-wire) is a thin, rigid wire that is suitable for use as a pin with this assembly.

Although the ends of the first pin 370 in FIG. 3 are described as sharpened and blunt for illustration purposes, it is understood that the ends do not need to be blunt and sharp. The sharpened end can be of any shape that allows the guide pin to penetrate and travel through bone. The blunt end can be of any shape to include sharp or blunt.

The guide assembly operates with a second pin which can optionally be provided as a part of the assembly. As is discussed below, the functions provided by the second pin in some embodiments, can be provided by common guide pins, guide wires, drill guides, borers or other surgical tools common to creating bone tunnels. The second pin structure is generally the same as the first pin however, the second pin does not necessary need the reference point as described below.

The guide assembly also has a reference facility. This reference facility provides a means to position one of the guide pins in a known relationship with other elements of the assembly, particularly another channel in the guide frame and eventually the other pin. The interrelationship of the reference facility with the guide assembly also provides a way to determine the length of one guide pin when compared to the other. FIG. 2 illustrates one embodiment of a frame reference point used as one element of the assembly reference facility. FIG. 3 illustrates a pin reference point that is used as another element of the assembly reference facility.

As shown in FIG. 2, the frame reference point is a point on the guide frame that cooperates with a guide pin reference point to define a relationship between the guide pin and the guide frame 220. In the embodiment shown in FIG. 2, the frame reference point 214A comprises the point on the distal side of the frame where the first pin channel 230 exits the body 210 of the guide frame 220. From this point, when the first guide pin is inserted into the first pin channel 230, portions of the pin can be viewed. Although a visual reference point is used as the reference point for this embodiment, other methods to create a point of reference with the pin reference point such as but not limited to mating connections between the guide frame and the first pin, visual methods such as through clear windows through the guide frame body, using the distal end of the body or other means of referencing points on the guide frame to points on the guide pin.

As shown in FIG. 3, the first pin further comprises a pin reference point. This reference point cooperates with the frame reference point so that the pin and the assembly are in a known relationship to each other. As shown in the embodiment in FIG. 3, the pin reference point 376 is a longitudinal reference point along the first pin 370 such as an etching or an outer recess such as a reduction in the radial profile such as a breakaway point. In the embodiment shown in FIG. 3, this pin reference point 376 is an etching along the pin. This etching allows a visual reference point to be aligned with the frame reference point that is the proximal exit point of the first guide pin channel. This cooperation of reference points and the pin channel puts points of the first pin 370 in a known location relative to other assembly elements. Although an etching is used in this embodiment to create a reference point 376 on first pin 370, other methods to create a point of reference are anticipated such as, but not limited to shoulders that limit the insertion of the pin beyond a certain point in the guide frame, buttons, hashes or other means of marking a point on the guide pin to be mated with the reference point on the guide frame.

In one embodiment, the pin reference point is an outer recess on the first guide pin that also serves the purpose of providing a breakaway point for the guide pin. Although not required, this allows the sharp end of the first guide pin to be broken off and removed from the surgical area.

As another part of the assembly reference facility, the first pin further includes a reference length defined as the longitudinal distance between the pin reference point and the pin dull end. Shown in FIG. 3 as 379, this reference length is defined as the length between the guide pin reference point 376 the dull end 374 of the first pin 370. The reference length 379 plus a setback length is generally equal to the length from the guide pin reference point 376 to the central point where the channel axes start to diverge.

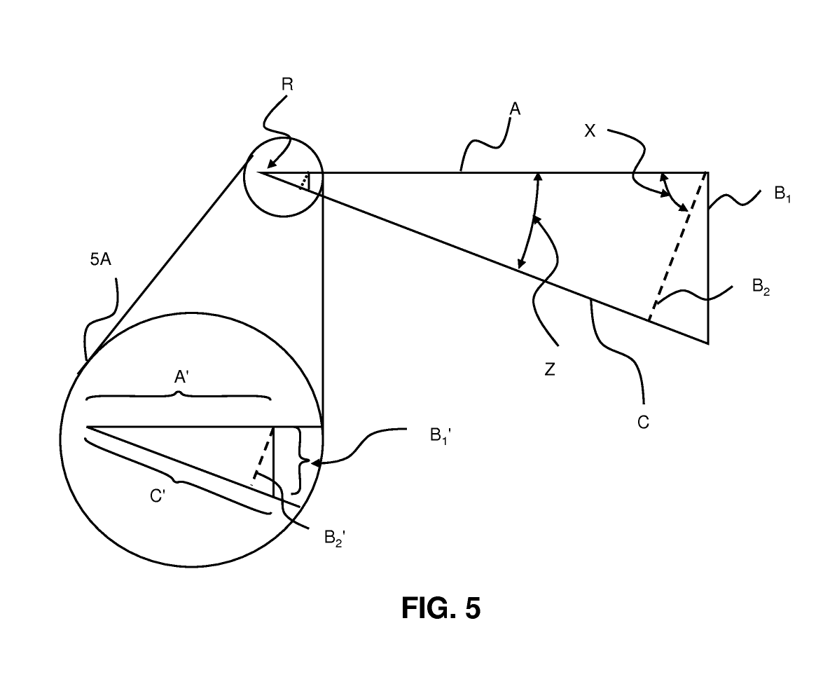

The reference facility utilizes the geometric relationship of a triangle as shown in FIG. 5. The angle Z is the angle between the sizes A and C of a triangle from a central convergence point R. The angle Z consistently defines the relationships of points on those two sides. Additionally, according to well known geometric principals, the sum of the squares of the legs of a right triangle will equal the square of the hypotenuse. As used in one embodiment, FIG. 5 illustrates the use of the these principles when sides A and Bsub1 are at a 90 degree angle to each other, the length of C is the square root of the sum of the side A squared and side Bsub1 squared. Also, given the right triangle of A, C and Bsub1, with the angle Z being known, Bsub1 can be determined as A multiplied by the tangent of Z. It is understood that similar relationships can be obtained by using side C and having it at a 90 degree angle from side Bsub1. It is also understood that given the angle of Z, the lengths of side can be determined if given other triangle dimensions or angles using common geometry and trigonometry principals. For example, if an angle X between side A and Bsub1 were not 90 degrees, but had a know value, together with the length of A and the angle Z, the other sides of the triangle could be determined such as a different side C (not shown) and the different side B as shown with dotted line Bsub2.

This geometrical relationship is applied to the guide assembly where the angle Z represents the angle the two channel longitudinal axes diverge from the central convergence point R. Side A represents the length of the first pin channel longitudinal axis for a distance of the first pin reference point to the central convergence point. This length includes the guide pin reference length plus the setback length. Side Bsub1 represents a relational distance point between a point on the second pin channel longitudinal axis at a 90 degree angle from the first guide pin reference point. Side C represents the second pin channel longitudinal axes for a length between the central convergence point and the point on the second pin channel axis at the relational distance point.

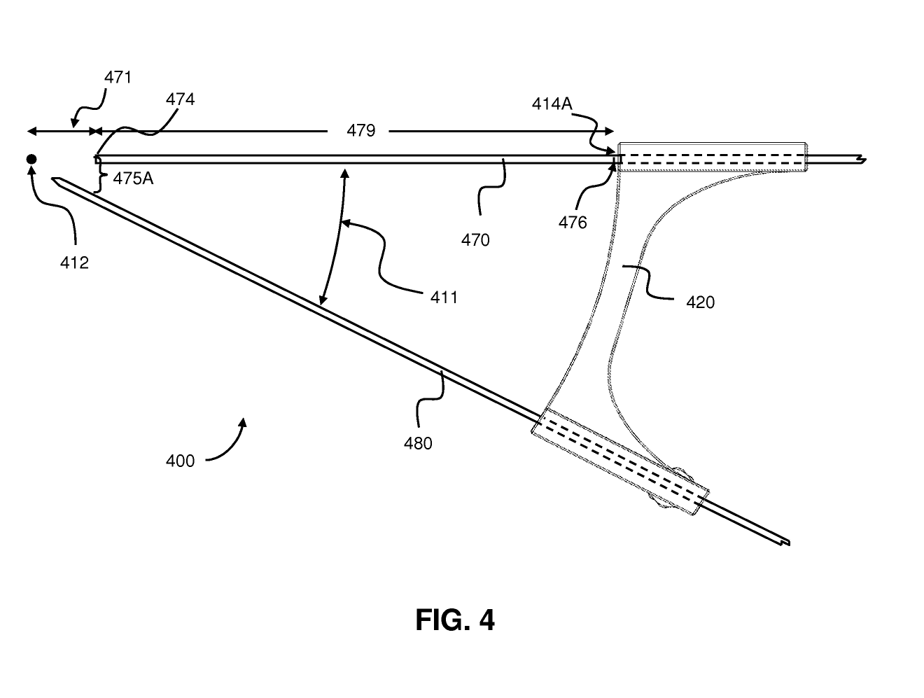

FIG. 4 illustrates one embodiment of the guide assembly 400 with the guide frame 420 and the first pin 470 comprising a guide pin. Although not necessarily part of the guide assembly 400, the second pin 480 is also shown. As can be seen when the assembly elements are used together in this embodiment, the cooperation of the tunnel angle 411, the pin reference point 476, the frame reference point 414A and the reference length 479 define a constant position of the pin blunt end 474 when the reference points are engaged. The position of the first guide pin blunt end 474 serves as a point of reference when the second pin 480 is put into the second channel of the guide frame. The longitudinal axis of the second pin tunnel, and therefore the second pin 480, can be maintained in the same relationship to the tip of first guide pin 470 and thus the angle of a bone tunnel created with the alignment of the second pin tunnel will maintain that relationship. This relationship creates a center-to-center relationship of the two longitudinal axes that correspond with the relationship of the center-to-center attachment sites in a double bundle ligament reconstruction.

As the geometric relationships illustrated in FIG. 5 create a reference facility using the assembly elements above, a similar relationship is created using the angle Z and the setback length of the first pin along an extended longitudinal axis and a square angle between the second pin and the extended axis of the first pin. As shown in the callout 5A in FIG. 5, this relationship, when a setback length is defined as A′, the length Bsub1′ defines the center-to-center positioning of the longitudinal axes of the first and second pins and tunnels at the angle Z. Side Bsub2′ defines a center-to-center position of longitudinal axes corresponding to the angle X from side A′. In an embodiment of ligament reconstruction surgery, given this relationship, the length Bsub1′ or Bsub2′ can define the center-to-center positioning of bone tunnels. The center-to-center position represented by side Bsub1′, Bsub2′ or other variations can be used with the assembly to account for the variable surface shapes where the tunnels exit such as on the intercondylar notch. When the size of the bone tunnels is known and their size is accounted for, this also defines the size of the bridge between the edges of the bone tunnel.

In some embodiments, the first guide pin can include features such as a pin retaining element that allow the guide pin to be more securely retained in the guide frame. For example, and not for limitation, the guide pin can include a recess that cooperates with the securing element to keep the guide pin from moving in and out of the pin tunnel. Other pin retaining elements include but are not limited to holes, springs or other means of mating the pin to the guide frame.

By using the above geometric relationship, it is possible to alter the guide pin reference length so that the setback length is also altered. This alteration in turn alters the center-to-center positioning of the tunnels axes at the tip of the first guide pin. As can be appreciated by those in the art, this allows a variety of center-to-center positions and therefore a variety of bone bridges to be created by altering the length of the guide pin reference length. Although not required, it is possible to create a kit of various length pins with varying reference lengths or single pins with multiple reference points.

In some embodiments, it is also possible to define the length of the second pin from a reference point on the guide frame. For example, FIG. 2 shows a second pin tunnel frame reference point 214B as the distal face of the guide frame 220 where the second tunnel exits the body 210. This additional reference may be helpful if there is a need to accurately position points on the second pin, for example, to keep a tip of second guide pin from extending too far beyond the bone when creating a tunnel. A reference facility can be defined similar to the first pin where the reference length is defined as a second reference length from a second frame reference point and this reference length is a second setback distance from the central convergence point. In this embodiment, the second pin could have a predefined reference point or the reference point could be defined by graduated markings on the guide pin and these measurements could be compared to a second frame reference point such as 214B.

### One Embodiment with Ligament Surgery

For one embodiment of the guide assembly for double-bundle ligament reconstruction, the center-to-center distance between the central longitudinal axes of the fixed channels and guide pins can be used to define the distance between the radial centers of the bone tunnels for attached the two ligament bundles. By using the assembly reference facility, by maintaining a consistent reference length, the center-to-center distance between these axes at the tip of the first pin is consistent. In turn, the center-to-center distance of the tunnels can define the end-to-end distance of a bone bridge between the two tunnels. Therefore, when the first pin is restrained in the first channel given a reference point of the first pin, the tip of the first pin can be at the center of one femoral attachment point, the angle Z of the second pin in the second tunnel places the second guide pin in a proper relationship to create the second bone tunnel with a tunnel exit point to define the center of the second femoral attachment point.

One embodiment of the guide assembly for ligament reconstruction is shown in FIG. 4 where the relationship of the blunt end 474 of the first pin 470 and the second pin 480, and the resulting center-to-center distances are illustrated. In this embodiment, for the femoral attachment site of an ACL ligament, the two longitudinal axes diverge at an angle 411 of about 20 to 50 degrees, or preferably about 20 to 40 degrees or more preferably about 30 degrees about the central point 412. The reference length 479 of the first pin 470 is in a range of about 10 to 23 cm, preferably about 12 to 18 cm or more preferably about 15.24 cm. In this embodiment, the length of the setback 471 is in a range of about 3 to 51 mm, or preferably about 17 to 28 mm or more preferably about 21 mm. In this embodiment, when the first pin 470 is engaged with the frame reference point 414A, the center-to-center distance, shown as 475A, of the tunnel longitudinal axes at the blunt end 474 of the first guide pin range from about 2 to 30 millimeters (mm), or preferably about 10 to 16 mm or more preferably about 12 mm.

A similar guide frame can be used for double-bundle PCL reconstructions. The same geometric relationships exist, however, the angle between the first and second pin tunnel is in a range of about 30 to 60 degrees, or preferably about 40 to 50 degrees or more preferably about 45 degrees about the convergence point. Additionally, because of the different dimensions of the PCL attachment site, a greater center-to-center distance range can be used. It is also understood that one guide can be configured to include both the ACL and the PCL guide angles for the second guide pin tunnel.

When used with common bone boring means, tunnels are typically created with a radial diameter in the range of about 4 to 15 mm or preferably about 8 to 10 mm. When used with an embodiment of the guide assembly described above for an ACL reconstruction, this generally creates a bridge between the edge of the tunnels in the range of about 1 to 10 mm, or preferably about 2 to 6 mm or more preferably 4 mm. Guides for PCL reconstruction are more likely to create bridges between the edge of the tunnels in the range of about 4 to 10 mm, or preferably about 6 mm.

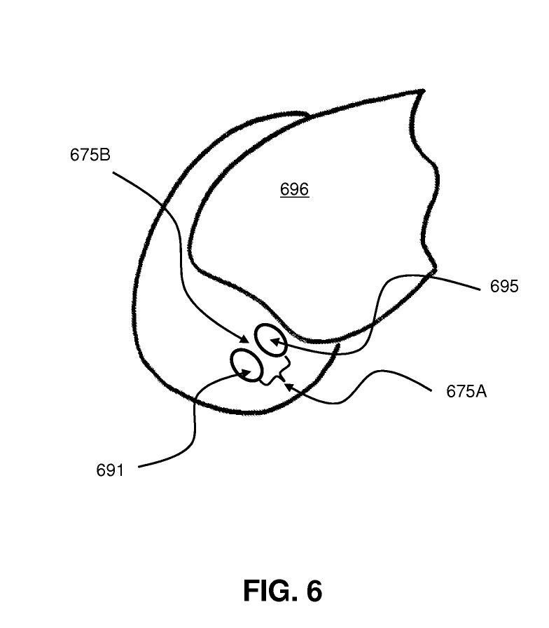

The results of one embodiment of the guide assembly when used with typical means to create bone tunnels for the femoral attachment of a PCL using a double-bundle procedure is shown in FIG. 6. The illustrative embodiment shown in FIG. 6 shows a cross-section view of a femur 696 having had two bone tunnels 691 and 695 created with the use of the guide assembly. In this embodiment, the assembly comprises a guide pin reference length of about 15 cm, a convergence angle of about 45 degrees and a set back of about 14 mm, which created a center-to-center tunnel distance, marked by 675A, between the center of the two bone tunnels 691 and 695 of about 14 mm, two tunnel radii of about 4 mm each and a bone bridge, marked as 675B, of about 6 mm.

By utilizing different reference lengths, correspondingly different setback lengths and center-to-center distances at the first guide pin tip can be obtained. When used in an outside-in technique, this center-to-center distance can be obtained by properly positioning the tip of the first guide pin and without the need to visually position the second tunnel or to position equipment in the area between the femur and the tibia. Corresponding to this feature, one embodiment of the guide assembly can comprise a first pin having several different predetermined reference points that correspond to predetermined bone bridge dimensions.

Thus for ACL and PCL repairs, the guide assembly provides tunnel centers at a known distance apart. Furthermore, the ability to rotate the second channel about the first channel, the position of the second tunnel can be adjusted to accommodate bundle positioning and surgical obstacles or special situations.

Although a guide frame with a solid body has been illustrated and described in this embodiment, it is recognized that configurations of the guide assembly that have properly aligned fixed recesses, channels, holes or other guide means can function as a guide frame to receive the pins and provide the relative angle between channels and provide a frame reference point.

Although a single body is illustrated in the above description, it is understood that a two-section guide frame or body can be utilized that is able to be secured in one configuration that maintains the relationships described above and also can have one configuration that allows the guide frame to expand and be removed when the guide pins are fixed in surgery. For example and not for limitation, the guide frame can have a first section that contains the first pin tunnel and a second section that contains the second pin tunnel. The sections can be connected through means such as clips, pins, button or other connection means that keep the proper geometric relationships of the tunnels and the and the guide frame. The section can also be disconnected, or expanded, so that the sections and tunnel can be expanded whereby when guide pins are in the tunnels and ends of the pins are set rigidly in a bone, the sections slide off of the diverging ends of the pins. This disconnection or expansion can be provided by disconnecting the connection means or providing expansion means such as telescoping fittings or slidable attachments that are held in place and released by buttons, clips, pins or other retaining means.

Cooperating with this assembly are the normal tools and methods used in ligament reconstruction surgery that are common to those in the art. These tools and methods include but are not limited to guide wires, reamers, drills and arthroscopic surgical tools.

### Reconstruction Guide Assembly in Operation:

The disclosed guide assembly is particularly useful in locating the position of tunnels in ligament reconstruction procedures. Although the following description of methods for operational use of the reconstruction guide assembly details the assembly's use with ACL reconstruction methods and connection to the femur, people skilled in the art understand the assembly can be used with PCL, ligament reconstruction on the tibia and other orthopedic procedures including but not limited to other double-bundle procedures in the rotator cuff.

During this procedure, which can be but not necessarily be an arthroscopic procedure, portals for the arthroscope and graft harvesting are made on the patient. Through these portals, the knee is examined by arthroscopic procedures and any observed minor defects or irregularities are taken care of.

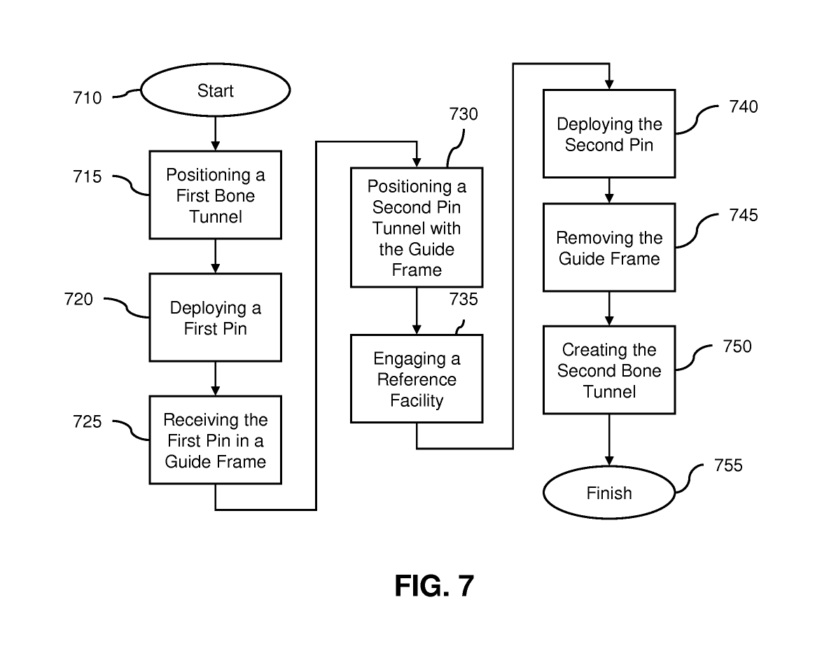

As shown in FIG. 7, the procedure starts with step 710 and proceeds to step 715.

Step 715 comprises positioning a first bone tunnel with a guide pin in the intercondylar notch of the femur. In a typical ACL procedure, this original bone tunnel can be positioned from the tibial tunnel extended up to and through the femur. This can be done with the knee in an extended position. This femoral tunnel may also be positioned by a common single tunnel ACL guide on the market or other standard techniques of locating ACL attachment sites as is common for a single bundle procedure. Another method of positioning a bone tunnel in a femur with a single bundle procedure is illustrated by U.S. Patent App. Pub. No. US2004/0193167 filed on Apr. 8, 2004 and entitled “TRANSVERSE SUSPENSION DEVICE” to Tucciarone et. al. is herein incorporated by reference in its entirety. Other methods of positioning the tunnel include, but are not limited to visually positioning it directly on the intercondylar notch.

The first pin is deployed with step 720 so that the pin reference length can define the distance from the intercondylar notch to the pin reference point. This is ensured by having the sharpened end of the first pin act as the leading end of the first guide pin through the femur. In one embodiment, the first pin travels through the tibial tunnel to the selected point on the femur. The first guide pin is inserted into the bone at the selected position and advanced into the femur. Once the guide pin emerges from the skin on the lateral thigh, a drill is then attached to its sharpened end and the pin is further retracted superior, just until the other tip, the guide pin dull end, can be seen flush on the lateral wall of the intercondylar notch.

Although not required, it is preferred that at this point that a femoral tunnel has not yet been started. This provides both a secure setting of the first guide pin in the bone structure of the femur at the intercondylar notch as well as the bone structure at the exit point of the pin from the femur. Similar consideration will be given on securing the ends of the guide pin when being used with other bones.

The guide assembly is then referenced to the ligament attachment site. This is performed by receiving the first pin into the first pin channel of the guide frame with step 725. If a drill is attached to the pin, the drill is removed and the guide assembly is passed down the first pin until it the pin and guide frame reference points are engaged.

The second tunnel is then positioned with the guide frame and the second pin with step 730. This positioning is performed visually by rotating the guide frame about the first pin so that the projected exit point of the second pin will correspond with the attachment sit of the second ligament bundle. Generally, although not required, the position of the second tunnel will be below the first pin (towards the knee joint) and at an angle of about 30 degrees posterior to the straight line extending from the knee to the foot of the leg when the leg is extended.

Once this proper rotational positioning is completed, the reference points on the first guide pin and the guide frame are aligned and engaged with step 735. This is performed by aligning the guide pin recess or etching with the guide frame reference point. While maintaining this alignment, the securing element is used to secure the guide pin relative to the guide frame. In one embodiment, the securing element is a set screw that is tightened onto the first guide pin. Although not required, in the embodiments shown in FIGS. 3 and 4, the first guide pin can be broken about a breakaway point to keep the users from encountering the sharpened end of the first guide pin.

With step 740, the second pin is deployed by inserting it through the second pin channel of the guide frame. The second pin is passed thru the guide frame to enter the soft tissue of the lateral thigh and then passed thru the bone to emerge inside the intercondylar notch along side the dull end of the first pin.

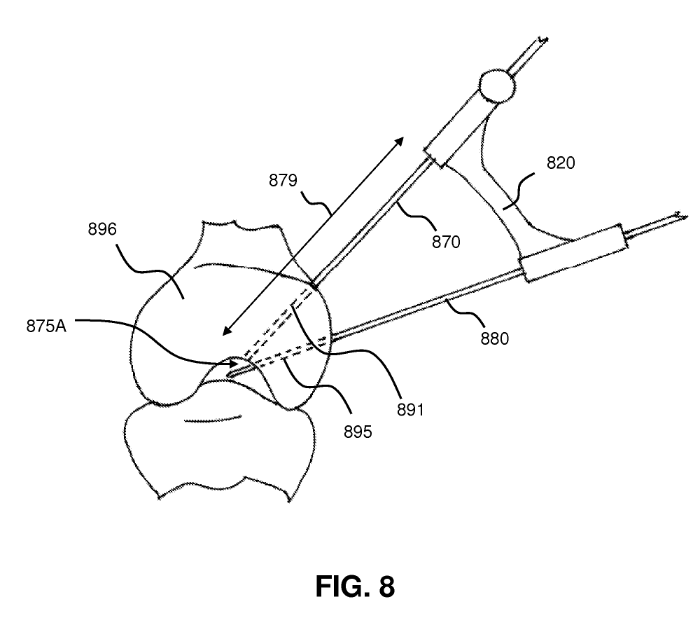

An illustration of these steps using one embodiment of the guide assembly when used with typical means to create bone tunnels for the femoral attachment of an ACL using a double-bundle procedure is shown in FIG. 8. In this embodiment, the first guide pin has been positioned in the femur 896, the guide frame 820 has been placed over the first pin 870 and the second pin 880 has also been positioned in the femur 896. The gap between the exit points of the first and second guide pin illustrate the center-to-center distance 875A of the two tunnels and this center-to-center distance, together with the tunnel bore size, determine the eventual size of the bone bridge.

The illustrative embodiment shown in FIG. 8 has reference length 879 of about 15 cm, a center-to-center tunnel distance 875A of about 12 mm, a setback of about 20.8 mm, tunnel radii of about 4 mm and a bone bridge 875B of about 4 mm.

After the second pin is exposed and the position of the tunnel can be viewed, the surgeon determines whether this positioning is satisfactory. If a different position of the second tunnel is desired, the second pin is retracted, the securing element is released, the guide frame is rotated and steps 735 and 740 are repeated until a satisfactory tunnel location is obtained. Generally, the surgeon is looking for the proper location of the exit of the second so that it is properly angled and creates a satisfactory bridge between the edge of the tunnels in the range of about 1 to 10 mm, or preferably about 2 to 6 mm. As discussed herein, the positioning of the guide pins, representing the centers of the bone tunnels, needs to accommodate the sizing of the final bone tunnel once drilled.

The guide assembly is then removed with step 745. In one embodiment of these methods, the guide is removed by forcing the first pin back into the intercondylar notch for a distance sufficient to attach a drill to the first pin bull end. The first pin can be forced by striking the sharpened end. In embodiments where the first pin is broken off at the reference point, the broken end of the guide pin can be placed on the end of the first guide pin and when struck, it will urge the first guide pin into the intercondylar notch.

In other embodiments of the method, the guide assembly can be removed by separating a two-section guide frame that allows the frame and the distance between the two pin channels to expand over the two pins so that the guide frame can be removed.

With step 750, now that the position of the second bone tunnel has been selected as running along the axis of the second guide pin, that tunnel is then drilled in a position along the longitudinal axis of the second pin. This can be done with the surgeon's drilling method of choice such as a cannulated reamer or drill bit being positioned over the guide pin to create the tunnel. The second bone tunnel can be created from an outside-in manner or from an inside-out manner. If needed, and the first tunnel has not yet been created, the first tunnel can be created in a similar manner. The first tunnel can be created from an outside-in manner or from an inside-out manner.

The method is finished with step 755 after which the ligaments can be positioned and secured in the bone tunnels.

The end result of one embodiment of this method used for positioning tunnels in a PCL reconstruction is a pair of bone tunnels with a center-to-center position that create a bridge at the exit points of the two tunnels as shown in FIG. 6. This illustration shows the positioning of the two tunnels where bone tunnels 695 and 691 are positioned by the first and second pin so that the center-to-center dimension of the tunnels comprises dimension 675A and the resulting bone bridges is represented by the dimension 675B which is the gap between the closest edges of the two bone tunnels.

These bone tunnels are then used in a double bundle procedures where ligaments are inserted through the tunnels and then secured.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1. A surgical guide assembly comprising:
a guide frame having a first pin channel and a second pin channel, each channel having a channel longitudinal axis;
the longitudinal axis of each channel is positioned at an angle to the other about a central convergence point;
a first pin capable of being received in the first pin channel; and
the assembly having a reference facility whereby placement of the first pin in the first pin channel predetermines the position of the longitudinal axis of the second pin channel.

2. The surgical guide assembly of claim 1 wherein the angle is between a range of about 10 to 50 degrees and the guide is used in positioning a bone tunnel in a double bundle ACL reconstruction.

3. The surgical guide assembly of claim 1 wherein the angle is between a range of about 30 to 55 degrees and the guide is used in positioning a bone tunnel in a double bundle PCL reconstruction.

4. The surgical guide assembly of claim 1 wherein:
the second pin channel is capable of receiving a second pin; and
the assembly is used in a double bundle cruciate ligament reconstruction whereby the second pin locates a bone tunnel.

5. The surgical guide assembly of claim 1 wherein:
the second pin channel is capable of receiving a second pin; and
the assembly is used in a double bundle cruciate ligament reconstruction whereby the second pin locates a bone tunnel created from an outside-in position.

6. The surgical guide assembly of claim 1 wherein the reference facility comprises:
a frame reference point;
a first pin reference point;
a first pin end; and
a predetermined reference length between the first pin reference point and the first pin end whereby the cooperation of the frame reference point with the pin reference point and the predetermined reference length of the first pin predetermines the position of the longitudinal axis of the second pin channel relative to the first pin end.

7. The surgical guide assembly of claim 6 wherein the angle is between a range of about 10 to 50 degrees and the guide is used in positioning a bone tunnel in a double bundle PCL reconstruction.

8. The surgical guide assembly of claim 6 wherein the angle is between a range of about 30 to 55 degrees and the guide is used in positioning a bone tunnel in a double bundle PCL reconstruction.

9. The surgical guide assembly of claim 6 wherein:
the first pin is a guide pin;
the second pin channel is capable of receiving a second guide pin; and
the assembly is used in a double bundle ligament reconstruction whereby the second guide pin locates a bone tunnel.

10. The surgical guide assembly of claim 6 wherein:
the first pin is a guide pin;
the second pin tunnel is capable of receiving a second guide pin; and
the assembly is used in a double bundle ligament reconstruction whereby the second guide pin locates a bone tunnel drilled from an outside-in position.

11. A reconstruction guide assembly comprising:
a guide frame having a first pin channel and a second pin channel, each channel having a channel longitudinal axis;
the longitudinal axis of each channel being positioned at an angle to the other;
a first rigid pin capable of being received in the first pin channel; and
the assembly having a reference facility whereby placement of the first rigid pin in the first pin channel predetermines the position of the longitudinal axis of the second pin channel.

12. The reconstruction guide assembly of claim 11 wherein the angle is between a range of about 30 to 55 degrees and the guide is used in positioning a bone tunnel in an arthroscopic double bundle ACL reconstruction.

13. The reconstruction guide assembly of claim 11 wherein the angle is between a range of about 10 to 50 degrees and the guide is used in positioning a bone tunnel in an arthroscopic double bundle PCL reconstruction.

14. The reconstruction guide assembly of claim 11 wherein the reference facility comprises:
a frame reference point;
a first pin reference point;
a first pin end; and
a predetermined reference length between the first pin reference point and the first pin end whereby the cooperation of the frame reference point with the pin reference point and the predetermined reference length of the first pin predetermines the position of the longitudinal axis of the second pin channel relative to the first pin end.

15. The reconstruction guide assembly of claim 14 wherein the angle is between a range of about 10 to 50 degrees and the guide is used in positioning a bone tunnel in a double bundle PCL reconstruction.

16. The reconstruction guide assembly of claim 14 wherein the angle is between a range of about 30 to 55 degrees and the guide is used in positioning a bone tunnel in a double bundle PCL reconstruction.

17. The reconstruction guide assembly of claim 14 wherein:
each channel longitudinal axis defined by the radial center of the respective channel;
the second pin channel is capable of receiving a second pin; and
the assembly is used in a double bundle cruciate ligament reconstruction whereby the second guide pin locates a bone tunnel.

18. The reconstruction guide assembly of claim 14 wherein:
each channel longitudinal axis defined by the radial center of the respective channel;
the second pin channel is capable of receiving a second pin; and
the assembly is used in a double bundle cruciate ligament reconstruction whereby the second pin locates a bone tunnel created from an outside-in position.

19. A method of locating a bone tunnel, said method comprising:
deploying a first pin with a longitudinal axis through a bone;
receiving the first pin in a first pin channel of a guide frame;
positioning a second pin with a longitudinal axis through a second pin channel of the guide frame;
deploying the second pin through a bone; and
locating a first and second bone tunnel by the longitudinal axes of the first and second pin.

20. The method of claim 19 further comprising:
the first pin having a first pin reference point;
the guide frame having a frame reference point; and
the step of receiving the first pin in a first pin channel of a guide frame further comprises engaging the first pin reference point with the frame reference point.

21. The method of claim 19 further comprising:
the first pin channel having a first longitudinal axis;
the second pin channel having a second longitudinal axis;
the longitudinal axis of each channel is positioned at an angle to the other about a central convergence point; and
the step of locating the first and second bone tunnel further comprises positioning each tunnel at the angle relative to the central convergence point.

22. The method of claim 19 wherein the first and second bone tunnels are used in a double-bundle ACL reconstruction procedure.

23. The method of claim 19 wherein the first and second bone tunnels are used in a double-bundle PCL reconstruction procedure.

24. The method of claim 19 the step of drilling the second bone tunnel further comprises drilling the bone tunnel from an outside-in position.

25. The method of claim 21 further comprising:
the first pin having a first pin reference point, a first pin end and a reference length between the first pin reference point and the first pin end;
the guide frame having a frame reference point; and
the step of receiving the first pin in a first pin channel of a guide frame further comprises engaging the first pin reference point with a frame reference point.
